(19) United States
(12) Patent Application Publication
(10) Pub. No.: US 2011/0264465 A1
(43) Pub. Date: Oct. 27, 2011

(54) Issuing Prescriptions from Standing Orders

(76) Inventor: Noel Lindsay, Ross, CA (US)

(21) Appl. No.: 13/044,377

(22) Filed: Mar. 9, 2011

Related U.S. Application Data
(63) Continuation-in-part of application No. 12/688,714, filed on Jan. 15, 2010.
(60) Provisional application No. 61/312,152, filed on Mar. 9, 2010.

(51) Int. Cl. G06Q 50/00; G06Q 10/00

(52) U.S. Cl. 705/3; 705/2

(57) ABSTRACT

Methods for processing a standing order by a server are disclosed. A standing order is received by a server. The standing order includes a plurality of prescription requirements. A request initiated by a user for a prescription product via a remote computing device is received. The request received from the remote device is processed by the server based on the prescription requirements. A prescription is automatically issued for the user based on the processed request.

### CROSS-REFERENCE TO RELATED APPLICATIONS

The present application is a continuation-in-part and claims the priority benefit of U.S. patent application Ser. No. 12/688,714 filed on Jan. 15, 2010, titled “Device for Automatically Issuing a Prescription for a Prescription Product,” which is hereby incorporated by reference in its entirety. The present application also claims the priority benefit of U.S. Provisional Patent Application Ser. No. 61/312,152 filed on Mar. 9, 2010, titled “Matching Patients with Healthcare Providers to Facilitate Prescriptions,”, which is hereby incorporated by reference in its entirety.

### BACKGROUND

Prescriptions are issued by authorized healthcare professionals for patients who require a product only available via prescription. Traditionally, a prescription is issued by the healthcare professional and then presented to a pharmacy or other service that can “fill” the prescription by providing the prescribed product to the patient. Typically, the pharmacy has a staff of trained professionals that can receive the prescription order and fill the order for the patient when the patient travels to the pharmacy.

In addition to the typical pharmacy, some issued prescriptions can be filled without the patient having to interact with trained professionals that provide the prescribed product. For example, a prescription service may fill a prescription for a patient and place the prescribed product in a secure location for the patient, such as a locker. These lockers can be placed in a convenient location for the patient, such as the patient's workplace.

Though prescription products can be picked up from convenient locations by a patient, the prescription itself still typically requires the patient to interact with a healthcare professional to have the prescription issued, most commonly through a face-to-face consultation.

### BRIEF SUMMARY OF THE INVENTION

Embodiments of the present invention provide systems, methods, and media for processing a standing order. The standing order may be processed to automatically generate a prescription by a healthcare professional for a prescription product. A prescription can be a set of instructions that govern a plan of care for a patient, and can be associated with a prescription product. Prescription products can be, for instance, a drug, a device, or any combination thereof. A prescription can be issued by a healthcare professional, for instance a physician, nurse practitioner, or dentist, any of whom can be a prescriber.

The present systems, methods, and media provide for automatically generating a prescription from a response to a questionnaire and may not involve physical inspection of the patient or other type of direct interaction between the prescriber and patient. The prescription can be automatically issued when a questionnaire response that fulfills a set of criteria provided by the prescribing healthcare professional is received. These criteria can be specified in or otherwise associated with the standing order to dispense a prescription product, for instance, a prescription anti-snoring device, when it has been determined that a threshold determination, such as all of the specified criteria, have been met.

A standing order may also direct an intermediary to prescribe a product under certain circumstances which may not solely require the interpretation of a questionnaire. For example, a doctor may give nurses a standing order to prescribe prescription-strength ibuprofen to a patient with a temperature that exceeds a specified threshold and who meets certain other criteria.

An embodiment may process a standing order by receiving a standing order by a server. The standing order may be associated with an authorized prescriber and include prescription requirements for a prescription product. A request for the prescription product may be initiated by a user and received via a remote computing device. The received request may be processed by the server, based on the prescription requirements. A prescription may automatically be issued for the prescription product for the user, based on the processed request.

An embodiment may include a computer readable storage medium having a program, wherein the program may be executable by a processor to perform a method for issuing a prescription. The method may receive a standing order including prescription requirements for a prescription product. A request may be received from a user for the prescription product, where the request may include a result of a health questionnaire. The prescription requirements of the standing order may be compared with the result of the health questionnaire. A prescription may be issued for the prescription product for the user from the standing order based on the comparison.

An embodiment may include a device for issuing a prescription may include a network interface, input interface, memory and a processor. The network interface may be configured to receive a standing order that includes prescription requirements for a prescription product. The input interface may be configured to receive a request from a user for the prescription product via user input to the device.

The request may include a result of a health questionnaire. The processor may execute instructions stored in the memory to compare the prescription requirements of the standing order with the result of the health questionnaire. The processor may also execute instructions to issue a prescription for the prescription product for the user from the standing order based on the comparison.

### BRIEF DESCRIPTION OF THE DRAWINGS

FIG. 1 is a block diagram of an exemplary networking environment in accordance with embodiments of the present invention.

FIG. 2 is a block diagram of an exemplary computing environment in accordance with embodiments of the present invention.

FIG. 3 is a block diagram of an exemplary standing orders engine in accordance with embodiments of the present invention.

FIG. 4 is a flow chart of an exemplary computer-implemented method for processing a standing order in order to automatically issue a prescription for a prescription product.

FIG. 5 is a flow chart of an exemplary computer-implemented method for validating a request for a prescription product in accordance with the methods discussed in the context of FIG. 4.

FIG. 6 is a flow chart of an exemplary method for matching patients with authorized prescribers.

### DETAILED DESCRIPTION

Embodiments of the present invention provide systems, methods, and media for processing a standing order provided by a healthcare professional authorized to prescribe to a patient. The standing order may indicate when it is appropriate to prescribe the prescription product, when it is not appropriate to prescribe the prescription product, when more information from the patient is needed, and the procedure that should be followed if more information is needed. The standing order may be processed to automatically issue a prescription to a patient by a healthcare professional for a prescription product. A prescription can be a set of instructions that govern a plan of care for a patient, and can be associated with a prescription product. Prescription products can be, for instance, a drug, a device, or any combination thereof. A prescription can be administered by a healthcare professional, for instance a physician, nurse practitioner, optician, or dentist. The present systems, methods, and media provide for automatically facilitating the issuing of a prescription from a response to a questionnaire answered by the patient, and may not involve physical inspection of the patient by the prescribing healthcare professional. In addition, the prescription can be automatically issued when patient data is received that satisfies or fulfills all or a certain portion of a set of criteria, or prescription requirements, for the standing order as provided by the healthcare professional. This patient data may be, for example, in the form of patient-provided responses to a questionnaire. These criteria can be associated with a standing order of a prescription product, which may be written by a healthcare professional. The prescription may therefore be issued in view of criteria set forth (the prescription requirements) by the healthcare professional in the standing order. Although the present disclosure focuses on anti-snoring devices, it is contemplated that this disclosure applies to automatically generating prescriptions for any prescription product, based on standing orders written by healthcare professionals who have the authority to issue a prescription.

A standing order may also direct an intermediary to prescribe a product under certain circumstances which may not solely require the interpretation of a questionnaire. For example, a doctor may give nurses a standing order to prescribe prescription-strength ibuprofen to a patient with a temperature that exceeds a specified threshold and who meets certain other criteria.

The systems, methods, and media described herein may make use of computerized questionnaires or other patient data received in response to an inquiry to a patient in order to determine whether the patient fulfills or satisfies required prescription criteria for a standing order of a prescription product. The questionnaires can be made available to patients via the Internet or other network to a client device associated with a patient, such as a desktop computer or a mobile device. The questionnaires may also be administered to a patient at a kiosk which includes a mechanism used to dispense the prescription product. The questionnaire may include a plurality of questions drawn to, for instance, health conditions, symptoms, and demographic information of the patient. A patient can be a user of the systems, methods, and media described who provides a questionnaire response. A questionnaire response may include a questionnaire received by the systems as disclosed herein with no questions answered, a portion of the questions answered, and all questions answered. In some embodiments, the questionnaire response may include comments provided by the patient. The patient may provide the questionnaire response via the Internet or another online network.

A healthcare professional may also be a user of the system who is an authorized prescriber of a prescription product. The authorized prescriber can be associated with a standing order which includes prescription requirements of a prescription product. The standing order can be satisfied upon receiving a questionnaire response in accordance with prescription requirements, as is described more fully below, and the prescription product can be dispensed pursuant to a prescription issued in accordance with a standing order. In some embodiments, the authorized prescriber may manage one or more standing orders associated with one or more prescription products. A prescription product may likewise, have several authorized prescribers.

Though the following discussion exemplifies the use of questionnaires in automatically generating prescriptions for prescription products, further applications and variations will become apparent to one skilled in the art upon review of this disclosure, including the use of data provided by various types of biometric sensors. In some embodiments, additional information may be received from a patient at a kiosk, vending machine, or other device. For example, a kiosk may have a blood pressure cuff and a thermometer. The system described herein may utilize the data from these and other sensors to prescribe products per one or more standing orders.

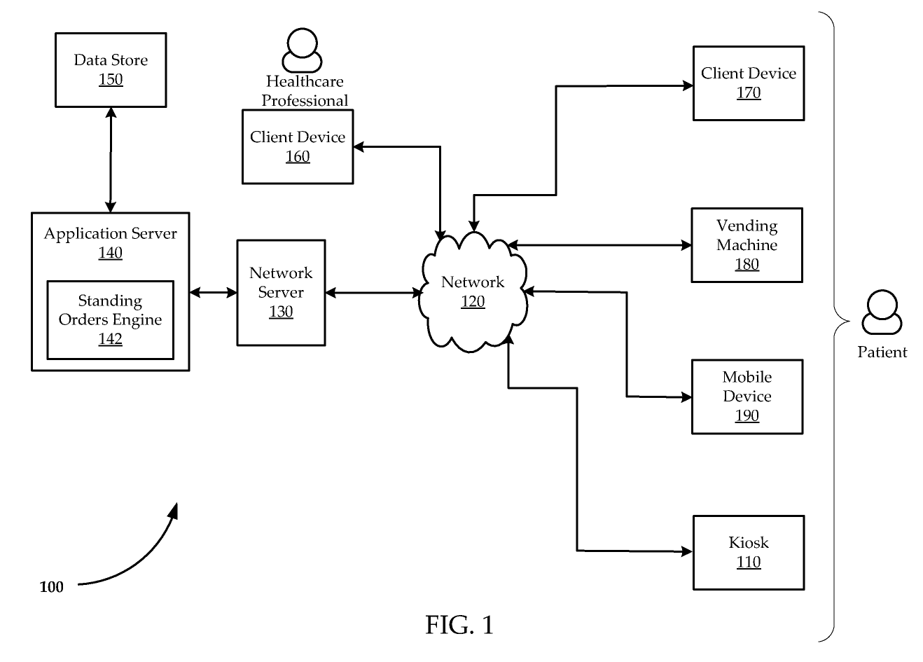

FIG. 1 is a block diagram of an exemplary networking environment 100 for facilitating the prescription and sale of a prescription product from an authorized prescriber to a patient. In the illustrated embodiment the networking environment 100 includes kiosk 110, network 120, network server 130, application server 140 with standing orders engine 142, data store 150, client devices 160 and 170, vending machine 180, and mobile device 190. In some embodiments, the client devices 160 and 170 may be implemented as computers having a processor that runs software stored in memory. The software may include network browser applications (not shown in FIG. 1) configured to render content pages, such as web pages, from network server 130.

The kiosk 110 may include a user interface, a payment device, a display device, a kiosk database, a network interface, a biometrics input device, a questionnaire engine, a processing engine, a prescription generation engine, and a mechanism to dispense prescription products, which can be in communication with each other and with the network 120 via a bus (elements not shown in FIG. 1). The kiosk 110 may also include one or more processors configured to execute software such as the standing orders engine 142, stored in memory within the kiosk 110. The kiosk 110 is further discussed in U.S. patent application Ser. No. 12/688,714, an application that is incorporated by reference herein and to which the present application claims a priority benefit.

The networking environment 100 illustrated in FIG. 1 is exemplary, and in embodiments may include more or less elements than those depicted in FIG. 1. As described in more detail below, the methods described herein can be executed by one or more of elements 110-190. The patient may interface the standing orders engine 142 via the client device 170, the vending machine 180, the mobile device 190, and/or the kiosk 110. For the purposes of clarity, the following disclosure typically refers to patient's use of the client device 170, although it is contemplated that any of the aforementioned devices may accept patient input and interface with the standing orders engine 142. For all figures mentioned herein, like numbered elements refer to like elements throughout.

The network 120 can be any type of network, including but not limited to the Internet, LAN, WAN, a telephone network, and any other communication network that allows access to data, as well as any combination of these. In some embodiments, the network 120 is coupled to the kiosk 110, the client devices 160 and 170, the network server 130, the mobile device 190, and the vending machine 180 as shown in FIG. 1. Additionally, the network 120 can be directly coupled to the application server 140 (not shown in FIG. 1). The networking environment 100 is exemplary and not limited to what is shown in FIG. 1.

Client devices 160 and 170 can be any computing device, including, but not limited to desktop computers, laptop computers, mobile devices, and portable digital assistants (PDAs). Each of the client devices 160 and 170 can communicate with a web service provided by network server 130 and application server 140 as well as with kiosk 110 and other elements of the networking environment 100 over network 120.

The mobile device 190 communicates with the application server 140 over the network 120. The mobile device 190 may include a memory, processor, and display, and store an executable application. The executable application may be executed by the processor to implement questionnaires, place a patient in communication with a healthcare professional (e.g., via chat, telephone, or email), receive input and provide output through the mobile device, and perform other functionality, such as the communication functionality described with respect to a kiosk. The mobile device 190 may be implemented as a mobile phone, PDA or other mobile device.

The vending machine 180 may receive input from a patient and communicate with the application server 140 via the network 120. The vending machine 180 may include a memory, processor, and display, in addition to a dispensing device. The vending machine 180 may additionally store an executable application, which may be executed by the processor to place a patient in contact with a healthcare professional, display an advertisement for a prescription product, dispense a prescription product and/or perform other functionality. In some embodiments, a vending machine may communicate with the application server 140 to confirm patient identification information received from a patient at the vending machine 180. The vending machine 180 may also dispense a prescription product based on information received from a patient and/or data received from the application server 140, such as for example identification of a prescription product to dispense to the patient.

The application server 140 can communicate with the network server 130 and the data store 150. It will be apparent to one skilled in the art that the embodiments of this invention are not limited to any particular type of server and/or database. In some embodiments, the servers mentioned herein are configured to control and route information via the network 120 or other networks (not shown in FIG. 1). The servers herein may access, retrieve, store and otherwise process data stored on any of the databases mentioned herein.

The data store 150 can store patient information such as government identification numbers, credit card numbers, biometric data and account information. The data store 150 can store any additional data, including, but not limited to, questionnaires, questionnaire responses, standing orders, issued prescriptions, prescription templates, contact information for patients and healthcare professionals, and electronic communication between healthcare professionals and patients. The data store 150 can also store historical action logs associated with server activity, e.g. date and time information associated with when questionnaires responses are received from a client device 170, when prescriptions were issued, and when prescription products were dispensed to the patient.

As described, embodiments may include more or less elements in the networking environment 100, and the networking environment 100 is configured to serve these elements. For example, the network server 130 may provide a questionnaire via the network 120 to a plurality of client devices 160 and 170 despite only two clients shown in FIG. 1. The network server 130 may also provide a questionnaire via the network 120 to a plurality of kiosks 110, mobile devices 190 and vending machines 180 despite only one client shown in FIG. 1.

In some embodiments, the networking environment 100 may allow healthcare professionals (authorized prescribers) to provide prescriptions issued on their behalf via the standing orders engine 142. As shown in FIG. 1, a healthcare professional may, via the client device 160 or the kiosk 110, communicate with the application server 140 via the network 120. A healthcare professional may provide prescriptions by writing a standing order for a prescription product.

A healthcare professional may identify himself or herself to the standing orders engine 142 via, for instance, a professional license code, biometric identifier, or any combinations thereof. The standing orders engine 142, after identifying the healthcare professional, may provide options for which prescription products (e.g. prescription drugs, prescription devices, and any combinations thereof) for which the healthcare professional may generate a standing order. These options can be displayed. The healthcare professional may select one or more prescription products for which to generate a standing order.

Upon selection of a prescription product by the healthcare professional, the standing orders engine 142 can be executed to prepare a questionnaire based on prescription requirements. Prescription requirements can be defined by the healthcare professional based on the questions selected or designated by the healthcare professional. In addition, the standing orders engine 142 can be executed to provide mandatory questions to be asked in the questionnaire. Mandatory questions may reflect prescription requirements of a manufacturer of the prescription product. For instance, in the case of a standing order for a prescription anti-snoring device, a question that differentiates central sleep apnea patients from ordinary snorers can be a mandatory question, in order to determine whether the patient qualifies for the prescription anti-snoring device. The standing orders engine 142 can be executed to provide assistance in generating the questionnaire, for instance, by querying historical patient data to correlate various answers to questions that have been asked in the past with various clinical outcomes. Thus, a questionnaire prepared by a healthcare professional via the standing orders engine 142 may reflect that healthcare professional's evaluation of the importance of various factors in determining whether to dispense the prescription product to the patient.

The healthcare professional, upon creating the questionnaire, may specify questionnaire responses that, if received from a patient associated with the client device 170, would satisfy the standing order and thus warrant the automatic issue of a prescription. For instance, in the case of a prescription anti-snoring device, a dentist may establish that a patient who provides questionnaire responses indicating that she is at least eighteen years of age and has no history of temporomandibular disorders satisfies that dentist's standing order. The healthcare professional may also define questionnaire responses that, if received from a patient, would not warrant automatic issue of a prescription, but instead, would require additional information from the patient, beyond what could be obtained through the questionnaire, potentially requiring follow-up by the prescriber or other intermediary. The standing orders engine 142 may also provide assistance to the healthcare professional for determining which questionnaire responses satisfy the standing order. The healthcare professional may provide questionnaire responses for some or all of the questions in the questionnaire.

After creating the questionnaire and defining the methodology for evaluating responses, the standing order can be generated and stored in the data store 150. Generating the standing order may involve associating data and/or information to the standing order. Expiration data, such as an expiration date, may be associated with the standing order. For instance, a standing order may be designated as valid by the standing orders engine 142 for a period of time, such as six months, and deactivated upon expiration. Additionally, the identification information provided by the healthcare professional may be stored in association with the generated standing order. Thus, the healthcare professional may, upon generation of a standing order, be identified by the standing orders engine 142 as an authorized prescriber of the prescription product.

In some embodiments, the standing orders engine 142 may process a payment while generating the standing order, and store the corresponding payment information in the data store 150. The payment accepted by the standing orders engine 142 may correspond to a purchase of a number of units of a prescription product for which prescriptions can be issued by the standing orders engine 142. Therefore, an inventory of prescription products may be generated in association with the standing order, and corresponding data and/or information may be stored in association with the standing order in the data store 150.

Upon generation of the standing order, the questionnaire may be made available to patients via the networking environment 100. A patient at the kiosk 110 may transmit a patient request for a prescription product to the application server 140 via the network 120. Upon receipt of the patient request at the application server 140, the application server 140 may transmit a questionnaire to the kiosk 110. The questionnaire may correspond to one or more standing orders stored in the data store 150. The patient may communicate a user response to questions in the questionnaire to the kiosk 110 via a user input interface. For instance, the questionnaire may include questions for determining the patient's height and/or weight, and the patient may provide a response indicating the corresponding information. The questionnaire can be provided for display on a display device associated with the kiosk 110, and the patient may communicate questionnaire responses to the kiosk 110 via a user interface (display device and user interface not shown in FIG. 1). The questionnaire response can be communicated via the network 120 to the network server 130 and to the standing orders engine 142 of the application server 140.

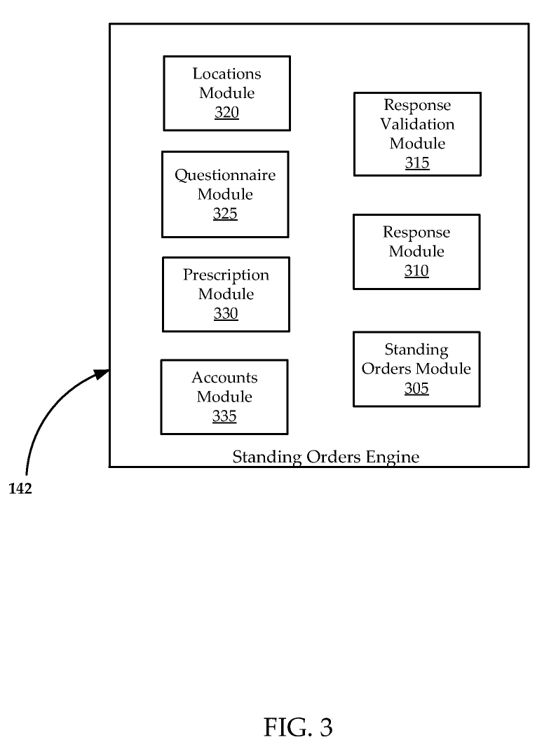

The patient request, including the corresponding questionnaire and questionnaire response can be evaluated and/or otherwise processed by the standing orders engine 142, which is further discussed herein in the context of FIGS. 3-5. As part of this evaluation, the standing orders engine 142 can be executed to access the data store 150 for prescription requirements and evaluate the questionnaire response based on the prescription requirements. Prescription requirements may include, for example, criteria that patients may, should, and/or must fulfill in order to qualify for a prescription. For instance, a prescription product such as a prescription anti-snoring device may require that a patient be older than eighteen years of age, not have full dentures, not have loose teeth, etc. The prescription requirements can be specified by and/or associated with a standing order for the prescription product. Standing orders can be further associated with one or more authorized prescribers of the prescription product, such as a healthcare professional, as is described more fully herein.

In evaluating the questionnaire response, the standing orders engine 142 at the application server 140 can be executed to automatically determine whether the questionnaire response satisfies prescription requirements associated with a standing order stored in the data store 150. As is described in more fully herein, a standing order may include some or all prescription requirements. In some embodiments, a questionnaire response can be compared with one or more standing orders. If the questionnaire response provided by a patient matches any available valid standing order retrieved from the data store 150, a prescription can be issued pursuant to that standing order on behalf of the prescriber who created it. Alternatively, if a questionnaire response meets, for instance, one or more prescription requirements, the standing order can be satisfied. For example, for a prescription pain medication, a question may ask a patient to rate, on a scale of one to ten, their level of pain. The patient may provide a pain level of 8 when the threshold level of pain for the question set by the authorized prescriber in the standing order is 4 or greater, thereby satisfying the standing order.

If the standing orders engine 142 determines that the questionnaire response satisfies the prescription requirements of the standing order, then a prescription for the prescription product may be automatically issued. In addition, the prescription product can be immediately dispensed to the patient. In some embodiments, the standing orders engine 142 may provide computerized instructions to the kiosk 110 or to the client device 170 to issue and provide a prescription to the patient and/or dispense the prescription product from the kiosk 110 via a kiosk dispensing mechanism (not shown in FIG. 1). Alternatively, the standing orders engine 142 may issue and transmit and/or otherwise communicate the prescription to the patient at the kiosk 110 or client device 170 via the network 120. The prescription can be printed at the kiosk 110 in hardcopy and/or softcopy for the patient's records. The prescription may additionally be transmitted and/or otherwise communicated to the authorized prescriber associated with the satisfied standing order.

The standing orders engine 142 may also generate and transmit instructions regarding a personalized message on behalf of an authorized prescriber which may or may not be associated with the satisfied standing order, which may also be included with the prescription and/or the product and can be printed at the kiosk 110 or other computing device associated with the patient. Alternatively, the prescription and the personalized message may be emailed to the patient on behalf of the authorized prescriber. The personalized message may for example, confirm order of the prescription product and thank the patient for their purchase. The personalized message may, at the discretion of the authorized prescriber, include additional information such as inviting the patient to call their office if they have any questions or problems, and even inviting the patient to call their office to schedule an appointment regarding the prescription product, such as a free fitting. This additional information may also include inviting the patient to contact the company's prescription product support group with any questions, and combinations thereof.

In the event that the patient is not satisfied with the product, he or she may contact the authorized prescriber or contact the company's prescription product support group, depending upon the particular details which result in the patient being unsatisfied. For example, patients likely would contact the authorized prescriber with questions regarding pain, discomfort, problems with fitting and questions about usage, but likely would contact the company's support group to inquire about missing orders and to request exchanges or refunds. These distinctions may be outlined in the personalized message included with the prescription and/or product, or emailed by the company to the patient on behalf of the authorized prescriber at the time of purchase as well as in subsequent printed and electronic communications with the patient on behalf of the authorized prescriber, as described in more detail below.

Subsequent to the sale of the product, the standing orders engine 142 may also generate and transmit instructions regarding follow-up messages to be transmitted or personalized emails to be sent to the patient on behalf of an authorized prescriber which may, at the discretion of the authorized prescriber, invite the patient to contact his or her office to schedule a phone call or office visit to resolve any problems. The particular details of these follow-up messages, including the authorized prescriber on whose behalf the message is sent and the patients who receive them, may depend on a number of factors including the prescription product which is prescribed. For example, for a prescription anti-snoring device, follow-up messages may be different for each of the following scenarios which may occur. A dentist may only offer a free fitting or follow-up session to patients who live or work near their offices as a way to convert these patients into new general dentistry patients. A dentist who accepts patients from a very wide geographic region may be primarily motivated by the opportunity to earn money, and may not commonly offer free fittings or follow-up exams—or even telephone consultations, as doing so would be impractical for patients who are far away and it may be impossible to handle phone calls from such a large number of patients. In such a case, the follow-up message may direct patients to the company's prescription product support group, and support personnel may refer patients to the dentist for any issues involving ongoing pain, loss of jaw mobility, or any other medical or dental condition.

A health care professional, such as for example a dentist, may also purchase the product on a wholesale basis, maintain an inventory at their office, prescribe it to their existing patients, and dispense the product directly to those for whom he or she feels it would be appropriate. They may sell the product to their patients at the retail price, if doing so is permitted in their state of licensure, or they may give it to their patients for free and then charge for services related to the product such as an initial examination, fitting, or follow-up assessment. In such a case, the dentist may offer the prescription anti-snoring device to their patients as a convenient and cost-effective way to address their snoring, but also as a low-cost way to evaluate a patient's suitability and tolerance for using anti-snoring devices before prescribing more expensive, custom-fabricated devices. Similarly, a health care professional who already has the ability to fit patients with custom-fabricated devices may be motivated to obtain patients of the prescription anti-snoring device, as these patients may be willing to upgrade to custom-fabricated devices at some point in the future.

The company may send follow-up messages and/or emails on behalf of an authorized prescriber at various points in time to achieve specific objectives. For example, shortly after delivery of the product, the company could send a message or email on behalf of an authorized prescriber to see if the patient is having any problems, and if so invite the patient to contact the authorized prescriber and/or the company's support group. At a point in time when a product would be getting worn-out or depleted, the company could send a message to the patient on behalf of an authorized prescriber, offering to refill or replace the product. If the relevant prescription is still valid, this communication could make it known that there is no need to go through the questionnaire process again. At the authorized prescriber's discretion, they could also offer discount pricing. At various points in the future, the company could send messages or emails to patients, making offers on behalf of the authorized prescriber to provide additional services, as the authorized prescriber deems appropriate. For example, dentists may want to offer free or discounted dental check-ups in an effort to convert the recipients into general dentistry patients. Prior to expiration of a valid prescription, the company could send a message or email to the patient to advise them that this is the case, and offer to replace or replenish the product before it happens.

Upon generating the prescription, an inventory of the prescription product associated with the standing order can be updated. For instance, if a standing order associated with an authorized prescriber indicates ten units of a prescription product in inventory, the standing order can be updated to nine units of prescription product upon issue of the prescription. Alternatively, the standing order can be updated when the prescription product is dispensed to the patient for whom the prescription is issued. In some embodiments, the issued prescription can be stored in association with the standing order and inventory information in the data store 150.

In some embodiments, upon issuance of the prescription, the standing orders engine 142 may also transmit instructions that the prescription product be shipped directly to the patient from the company's prescription product fulfillment center on behalf of the authorized prescriber. In such an embodiment, the company may maintain a “virtual” inventory on behalf of each such authorized prescriber, which would be decremented as sales are made and automatically replenishes as needed by purchasing product at the wholesale price from the company. Upon shipment of the product, the company could then bill the patient on behalf of the authorized prescriber such as by charging the patient's credit card.

If the patient returns the product for a refund, the company may refund the full retail price of the product to the patient and debit the authorized prescriber's account for the difference between the retail and wholesale price. Alternatively, the company could refund the full retail price of the product to the patient, and debit the authorized prescriber's account for the full retail price, and add the returned unit(s) to the “virtual” inventory of the authorized prescriber.

In some embodiments, implementing a standing order may include permitting only a specified number of prescription product units to be prescribed pursuant to a given standing order. In some embodiments, a standing order may be implemented for an unspecified number of prescription product units to be prescribed pursuant to the given standing order.

If the questionnaire response satisfies the standing order, the standing orders engine 142 may generate a user account for the patient. Patient information can be stored in association with the user account in the data store 150. Patient information may include, but is not limited to patient contact information such as an e-mail address, telephone numbers, the patient request (including questionnaire responses provided by the patient and the corresponding questionnaire), government identification numbers, and/or mailing addresses, authorized prescriber information, a softcopy of the prescription, billing information such as credit card information, and an indication as to whether the prescription product has been dispensed to the patient. The patient may access the user account at a future time, and may protect the user account via, for instance, a username and password or other credential.

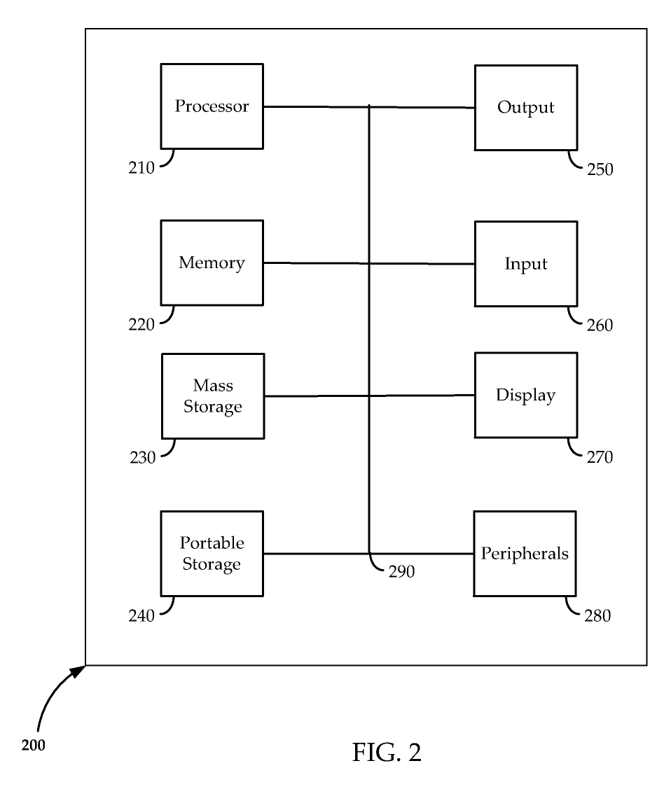

FIG. 2 is a block diagram of an exemplary computing device for automatically issuing a prescription for a prescription product in accordance with embodiments of the present invention. In some embodiments, the exemplary computing device of FIG. 2 can be used to implement portions of the client devices 160 and 170, the network server 130, the application server 140, the data store 150, the kiosk 110, the mobile device 190, and the vending machine 180.

The computing system 200 of FIG. 2 includes one or more processors 210 and memory 220. Main memory 220 stores, in part, instructions and data for execution by processor 210. Main memory 220 can store the executable code when in operation. The system 200 of FIG. 2 further includes a mass storage device 230, portable storage medium drive(s) 240, output devices 250, user input devices 260, a graphics display 270, and peripheral devices 280.

The components shown in FIG. 2 are depicted as being connected via a single bus 290. However, the components can be connected through one or more data transport means. For example, processor unit 210 and main memory 220 can be connected via a local microprocessor bus, and the mass storage device 230, peripheral device(s) 280, portable storage device 240, and display system 270 can be connected via one or more input/output (I/O) buses.

Mass storage device 230, which can be implemented with a magnetic disk drive or an optical disk drive, is a non-volatile storage device for storing data and instructions for use by processor unit 210. Mass storage device 230 can store the system software for implementing embodiments of the present invention for purposes of loading that software into main memory 220.

Portable storage device 240 operates in conjunction with a portable non-volatile storage medium, such as a floppy disk, compact disk or Digital video disc, to input and output data and code to and from the computer system 200 of FIG. 2. The system software for implementing embodiments of the present invention can be stored on such a portable medium and input to the computer system 200 via the portable storage device 240.

Input devices 260 provide a portion of a user interface. Input devices 260 may include an alpha-numeric keypad, such as a keyboard, for inputting alpha-numeric and other information, or a pointing device, such as a mouse, a trackball, stylus, or cursor direction keys. Additionally, the system 200 as shown in FIG. 2 includes output devices 250. Examples of suitable output devices include speakers, printers, network interfaces, and monitors.

Display system 270 may include a CRT, a liquid crystal display (LCD) or other suitable display device. Display system 270 receives textual and graphical information, and processes the information for output to the display device.

Peripherals 280 may include any type of computer support device to add additional functionality to the computer system. For example, peripheral device(s) 280 may include a modem or a router.

The components shown in the computer system 200 of FIG. 2 are those typically found in computer systems that can be suitable for use with embodiments of the present invention and are intended to represent a broad category of such computer components that are well known in the art. Thus, the computer system 200 of FIG. 2 can be a personal computer, hand held computing device, telephone, mobile computing device, workstation, server, minicomputer, mainframe computer, or any other computing device. The computer can also include different bus configurations, networked platforms, multi-processor platforms, etc. Various operating systems can be used including Unix, Linux, Windows, Macintosh OS, Palm OS, and other suitable operating systems.

FIG. 3 is a block diagram of an exemplary architecture of a system in the form of a standing orders engine 142 for processing and managing standing orders. Specifically, FIG. 3 as shown depicts further details of the standing orders engine 142, which is also shown as part of the networking environment 100 of FIG. 1. As such, patients, healthcare professionals, and authorized prescribers may communicate with the standing orders module 142, for instance via client devices 160 and 170, via the network 120.

The standing orders engine 142 may include one or more modules for performing one or more methods as described herein. In the embodiment illustrated in FIG. 3, the standing orders engine 142 includes a standing orders module 305, a response module 310, a response validation module 315, a locations module 320, a questionnaire module 325, a prescription module 330, and an accounts module 335. Each module of the standing orders engine 142 can be implemented by software which can be stored in memory and executed by a processor, and/or in specialized hardware designed to implement the specific modules. Each of the modules 305-330 may communicate with one another. Although various modules may be configured to perform some or all of the various steps described herein, fewer or more modules may be provided and still fall within the scope of various embodiments.

The standing orders module 305 may be executed to generate and process standing orders. When generating a standing order for a prescription product, a health professional may interface with the standing orders module 305 via the client device 160. As such, a user request, e.g., a prescriber request, received at the standing orders engine 142 may be directed to the standing orders module 305 for processing. The prescriber request may include validation information, for instance biometric identifiers, professional license identifiers, user account credentials, address and contact information, and the like, which are processed at the standing orders module 142.

The standing orders module 305 may process the prescriber request in accordance with the one or more of the aforementioned identifiers. For instance, the standing orders module 305 may locate credentials in the prescriber request for a user account, e.g., a prescriber account. The standing orders module 305 may transmit the credentials to the accounts module 335 in order to determine whether the healthcare professional is recognized as an authorized prescriber of prescription products. If the health professional is recognized as an authorized prescriber, the standing orders module 305 may then validate the prescriber and grant access to a previously established prescriber account. The authorized prescriber may then be guided by the standing orders module 305 in order to generate a standing order.

However, if the prescriber request does not include user account credentials, the standing orders module 305 can be executable to establish a prescriber account at the accounts module 335. As such, the health professional may be validated by the standing orders module 305. Validation of the prescriber may include verifying identity information, for instance, biometric identifiers and professional license information. Upon validation by the standing orders module 305, the health professional (now authorized prescriber) may then be granted access to the standing orders engine 142 and may generate a standing order by the standing orders module 305.

The standing orders module 305 may provide a series of graphical user interfaces (GUIs) for display by the client device 160 in order to guide the healthcare professional in generating a standing order (GUIs not shown in FIG. 3). For instance, upon processing the professional license information, the standing orders module 305 may provide to the client device 160 a selection of prescription products for which the authorized prescriber may generate a standing order. Upon receiving a selection of one or more prescription products via the graphical user interface, the standing orders module 305 may prompt the authorized prescriber to generate an inventory to be associated with the standing order. The authorized prescriber may, via the graphical user interface, select any number of units of the prescription product to be designated as inventory associated with the standing order. The standing orders module 305 may process these selections and thereby generate a standing order. The standing order may be stored in association with the prescriber account in the data store 150.

In some embodiments, the standing orders module 305 may generate the standing order upon processing a payment. For instance, the authorized prescriber may, based on the selected number of units of prescription product in the inventory, request to purchase a portion of the inventory via user input to the client device 160. As such, the standing orders module 305 is executable to receive payment information, for instance, credit or debit card information and other forms of payment known in the art.

The standing orders module 305 may allow the authorized prescriber to manage payments associated with the standing order. For instance, an authorized prescriber may interface the standing orders engine 142 at the standing orders module 305 and add or remove units of prescription product to one or more standing orders. The standing orders module 305 may then update the standing order and request payment from the authorized prescriber based on the updated standing order. Further, the standing orders module 305 may make payments to an authorized prescriber when prescriptions are issued from the standing order. These payments can be managed and/provided for display on the client device 160 by the standing orders module 305. In some embodiments, the prescribing process may be based on medical circumstances while the dispensing process requires a prescription and payment. Both the prescribing process and the dispensing process may be managed by the technology described herein, and may be linked or handled separately.

The present technology may issue a prescription in one or more of several ways. For example, the present technology may issue a prescription for the patient by printing prescription information at a vending machine, kiosk or other machine or device, communicating the prescription to a device via an SMS, MMS, or other data communication format, transmitting prescription information via email or facsimile, or some other communication method.

Additionally, issuing a prescription may include providing the patient with a prescription product associated with the prescription. The prescription product may be provided to the patient at a vending machine, kiosk, or pharmacy, or it may be shipped to the patient. When shipped, the prescription product may be shipped from a healthcare provider or another entity in communication with the healthcare provider.

In some embodiments, a prescription product inventory may be maintained for one or more healthcare providers by a third party. The entity may maintain a virtual inventory for one or more healthcare providers, and may update the inventory based on changes authorized by the healthcare provider. The virtual inventory may be a subset of the actual inventory of prescription products maintained by the entity. This subset of inventory would therefore be dedicated to be used (i.e., shipped) to fulfill the prescriptions issued by a particular healthcare provider per a standing order. In some embodiments, the actual inventory maintained by an entity may be less than the total virtual inventory maintained by the healthcare providers that store prescription products with the entity.

In some embodiments, the healthcare provider would provide payment for a beginning inventory. As prescription products were prescribed by the healthcare provider, the inventory would decrease. As the healthcare provider added additional inventor (for example, through payment) or prescription products were returned, the inventory would increase.

Alternatively, the virtual inventor may be associated with a limit to a standing order provided by a healthcare professional. Hence, if a standing order was limited to twenty prescription products, the healthcare professional's virtual inventory would begin with twenty prescription products.

Hence, in some embodiments, the present technology may be used to maintain a virtual inventory of prescription products on behalf of one or more healthcare professionals 160 of FIG. 1, wherein the healthcare professionals generate standing orders for prescriptions to be issued by standing orders engine 142. When the prescription is issued, the prescription order may be fulfilled by shipping a prescription product from a physical inventory location to the patient to which the prescription is issued. An entity, either human or automated, that controls the physical inventory location may be in communication with standing orders engine 142 and/or healthcare professional 160 to track, ship prescription products from, and add to a virtual inventory of prescription products associated with the healthcare provider.

In addition to generating standing orders, the standing orders module 305 may be executable to manage a plurality of standing orders. As discussed earlier, a healthcare professional can become an authorized prescriber of a prescription product when generating a standing order. The authorized prescriber may also generate a user account via the accounts module 335. Prescriber information can be stored in association with the user account in the data store 150. Prescriber information may include, but is not limited to prescriber contact information, such as an e-mail address, telephone numbers, identification numbers, and/or mailing address, authorized prescriber information, patient prescriptions associated with the authorized prescriber's standing order, billing information such as credit card information, and questionnaires and/or questionnaire responses associated with the authorized prescriber's standing order.

As such, the standing orders module 305 may interface and/or communicate with other modules at the standing orders engine 142. For instance, an authorized prescriber may request to review prescriptions issued to patients from the standing order. Thus, the standing orders module 305 may transmit instructions to the prescription module 330, which may access issued prescriptions from the data store 150 and provide the requested prescriptions for display. Also, for instance, an authorized prescriber may provide a location to the standing orders module 305. This location may, for instance, correspond to a geographical location where the authorized prescriber resides or is licensed to practice. As such, the standing orders module 305 may receive geographical information from the authorized prescriber by the client device 160, and transmit instructions to the locations module 320. The locations module 320 is further described herein.

An authorized prescriber may also wish to manage one or more questionnaires associated with the standing order by the standing orders engine 142. As such, the standing orders module 305 may provide one or more questionnaires for display by client device 160. An authorized prescriber may review the questionnaire and questionnaire responses associated with the standing order and edit her questionnaire and prescription requirements. Such edits may be communicated to the questionnaire module 325, which may store a new questionnaire in association with the authorized prescriber in the data store 150.

A patient may interface with the standing orders engine 142 when obtaining a prescription for a prescription product. The patient may transmit a request for the prescription product to the standing orders engine 142 by, for instance, the client device 170 shown in the context of FIG. 1. The request, e.g., the patient request, may be received by the standing orders engine 142 at the response module 310.

The response module 310 may, upon receiving the patient request, determine whether the patient is eligible for receiving an automatically issued prescription from the standing orders engine 142. Upon receiving the patient request from the client device 170, the response module 310 may parse and/or otherwise locate patient identifiers in the patient request. Such patient identifiers may include, but are not limited to, a patient name, biometric identifiers, patient location identifiers, and/or information relating to the requested prescription product, such as prescription requirements.

The response module 310 may compare the received patient request with previously received user requests, e.g., patient requests and prescriber requests, received by the standing orders engine 142. For instance, the response module 310 may formulate a search key from the patient identifiers and perform a search of issued prescriptions and/or previously received patient requests stored in the data store 150. A search of the data store 150 may return one or more previously stored patient requests. If the patient request matches a patient request stored in the data store 150, the patient may be considered ineligible to automatically receive a prescription for the prescription product. In some embodiments, the response module 310 may generate an alert to the client device 170, an authorized prescriber, and/or a system administrator (alert module not shown in FIG. 3).

If the patient is deemed eligible, the response module 310 may validate the patient. The response module 310 may then transmit instructions to the questionnaire module 325, which can be executed to select a questionnaire for the patient at the client 170. The questionnaire module 325 may determine which questionnaire to provide based on a plurality of factors. For instance, the questionnaire module 325 may randomly select a questionnaire from the data store 150. By randomly selecting a questionnaire, the questionnaire module 325 therefore randomly selects the associated standing order and associated authorized prescriber. Alternatively a patient may transmit a request that identifies a health professional who is an authorized prescriber of the prescription product. As such, the questionnaire module 325 may retrieve, from the data store 150, a questionnaire associated with the authorized prescriber. Thus, the patient, via selection of an authorized prescriber, selects the questionnaire.

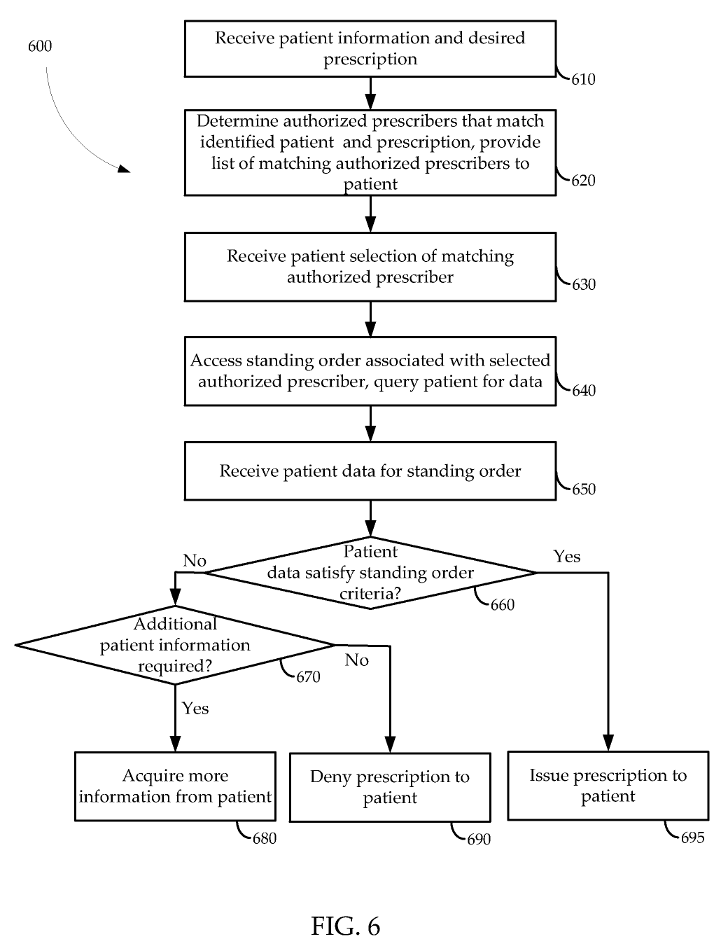

The questionnaire module 325 can be executed to select a questionnaire based on aspects of the standing orders stored in the data store 150. For example, two dentists, A and B, who are authorized prescribers, may each have a standing order for a prescription anti-snoring device stored in Data Store 150. A's standing order inventory may reflect a purchase of ten units of the prescription anti-snoring device whereas B's standing order inventory may reflect a purchase of twenty units. The questionnaire module 325, based on the higher number of units in B's standing order, may provide B's questionnaire for display at the client 170 in lieu of A's. In another example, a patient may enter a location identifier, for instance, a zip code, as a part of the patient request. If the zip code indicates a proximate location of the patient to A, the questionnaire module 325 may be configured to provide A's questionnaire to the patient in lieu of B's. As such, the questionnaire module may include one or more questionnaire rules which may be used to determine which questionnaire, and therefore, which authorized prescriber and standing order, is provided to the patient. These rules may include, but are not limited to number of units in standing order, seniority of the authorized prescriber in relation to others who prescribe the same prescription products, location of the authorized prescriber, and patient ratings and/or feedback of authorized prescribers. Instructions that govern the selection of questionnaires to be provided for display, such as those discussed in the examples above, may therefore be embodied in an algorithm executable by the questionnaire module 325. Further algorithms for selecting standing orders for patients are discussed in the context of FIG. 6.

A patient may provide questionnaire responses to the standing orders engine 142 via the network 120. Receiving questionnaire responses may include receiving biometric information from the patient, for instance, a fingerprint scan. Questionnaire responses can be received and processed by the questionnaire module 325, upon which the questionnaire responses and/or biometric information can be passed to the response validation module 315. The response validation module 315 may access the questionnaire responses provided by the authorized prescriber associated with the questionnaire. The response validation module 315 may, by determining whether the questionnaire response meets the prescription requirements, determine whether the questionnaire responses satisfy the standing order.

If the response validation module 315 determines that the questionnaire response satisfies the standing order, then the response validation module 315 may transmit instructions to the prescription module 330 to issue a prescription. The prescription module 330 may access a generic prescription template stored in the data store 150. Using patient information and information received from the questionnaire responses, the prescription module 330 may issue a prescription and provide the prescription to the patient via the network 120. Alternatively, if the authorized prescriber of the satisfied standing order has provided a prescription template, the prescription module 330 may retrieve the template from the data store 150 and issue a prescription using the same.

In issuing the prescription, the prescription module 330 may locate contact information of the authorized prescriber in the data store 150. The prescription module 330 may then transmit an electronic message including the prescription to the authorized prescriber, indicating that the patient's responses satisfied the prescription requirements specified in the standing order. In some embodiments, the questionnaire responses may be transmitted to the authorized prescriber in conjunction with the issued prescription.

If the response validation module 315 determines that the questionnaire response satisfies the standing order, then the response validation module 315 may transmit instructions to a payment module to receive payment from the patient (payment module not shown in FIG. 3). The prescription can be issued contingent upon receiving payment from the patient. Alternatively, payment can be received prior to dispensation of the prescription product, for instance, if the patient is at the kiosk 110. Payment can be received in the form of currency, such as coins and bills, credit card, debit card, and other forms of payment.

If the response validation module 315 determines that the questionnaire response satisfies the standing order, the response validation module 315 may transmit instructions to the accounts module 335 to generate a user account for the patient, e.g., a patient account. Patient information can be stored in association with the user account in the data store 150. Patient information may include, but is not limited to, patient contact information such as an e-mail address, telephone numbers, government identification numbers, mailing address, authorized prescriber information, questionnaire responses, the issued prescription, billing information such as credit card information, and an indication as to whether the prescription product has been dispensed to the patient. The patient may access the user account at a future time, and may protect access to the user account via, for instance, a username and password or other credentials.

In some embodiments, the response validation module 315 may, upon determining that the questionnaire response satisfies the standing order, transmit instructions to the response module 310. The response module 310 may then determine whether the standing orders engine 142 has previously issued a prescription to the patient. For example, the response module 310 may perform a search of the data store 150 based on patient information and/or account information. If the search yields one or more patient requests, issued prescriptions, and/or questionnaire responses associated with the patient account, then the response module 310 may execute instructions to deny issue of the prescription. The response module 310 may additionally generate and transmit the alert to a system administrator associated with the standing orders engine 142. Alternatively, the response module 310 may transmit the alert to an authorized prescriber associated with the previously issued prescription.

The standing orders engine 142, via the response module 310, may be configured to automatically issue a prescription for the prescription product in accordance with one or more prescription rules. For instance, for some prescription products, it may be typical to issue multiple prescriptions to a patient, and as such, multiple valid patient requests may be stored in the data store 150. Thus, as in the example presented above, the standing orders engine 142 may automatically validate the patient request and issue a prescription despite having retrieved multiple patient requests from the data store 150. The response module 310 may therefore execute a prescription rule that allows the standing orders engine 142 to automatically issue a prescription for a threshold number of patient requests. If the number of patient requests exceeds the threshold, then the response module 310 may execute instructions to deny the automatic issue of the prescription.

The response module 310 may store one or more prescription rules in the data store 150 and evaluate patient requests via a rules engine (rules engine not shown in FIG. 3). Prescription rules may be established by an authorized prescriber or an administrator of the standing orders engine 142 and stored in the data store 150. Exemplary prescription rules may include for instance the number of patient requests permitted per patient account per prescription product.

In some embodiments, the response validation module 315 may, upon determining that the questionnaire response satisfies the standing order, transmit instructions to the locations module 320. The locations module 320 may then determine a location at which to dispense the prescription product. In some embodiments, the locations module 320 may receive a location identifier, for instance, a zip code, from the response validation module 315 and/or the response module 310. The locations module 320 may then provide to the client 170 a list of dispensaries in proximate location to the zip code based on, for instance, a search of the data store 150. The dispensaries may include pharmacies, fulfillment centers, vending machines such as the vending machine 180, kiosks such as the kiosk 110, and/or offices of authorized prescribers.

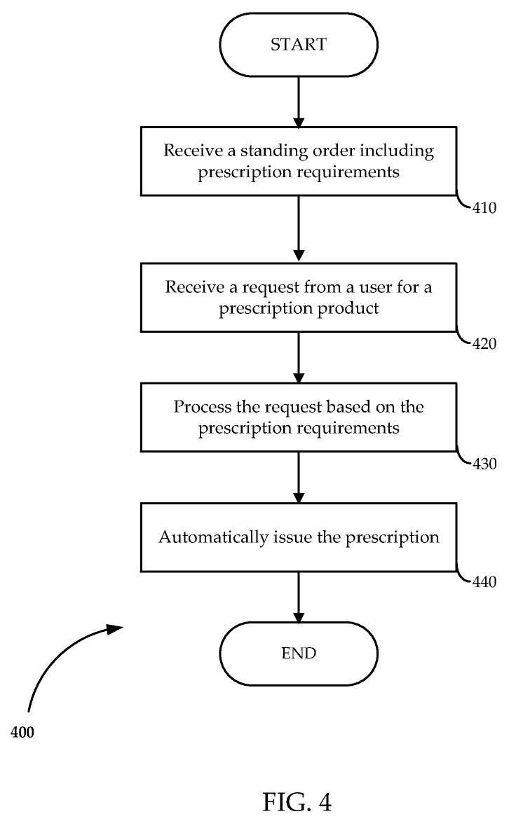

FIG. 4 is a flow chart of an exemplary computer-implemented method for automatically generating a prescription for a prescription product. The method 400 disclosed in FIG. 4 can be practiced in the networking environment 100 via elements discussed in the context of FIGS. 1-3 above.

In step 410, a standing order for a prescription for a product is received. This can be conducted via, for instance, input received by the standing orders engine 142 if the healthcare professional is at the client device 160. Upon generating a standing order, a healthcare professional can become an authorized prescriber of one or more prescription products after validation, as discussed above. The standing order may be generated based on prescription requirements selected by the authorized prescriber and/or the manufacturer of the prescription product. Selecting the prescription requirements may involve generating a questionnaire to be associated with the standing order, which may be made available to a patient at the client device 170. The standing order can be stored in the data store 150 for future retrieval by, for instance, the questionnaire module 325 and/or the standing orders module 305. In some embodiments, a user account, e.g. a prescriber account, can be generated by the accounts module 335 upon generation of a standing order. The standing order may be stored in association with the prescriber account in the data store 150.

In step 420, a request, e.g. a patient request, for a prescription product is received. The user request may correspond to a selection for a questionnaire, a questionnaire response, or any combination thereof. The questionnaire response can be provided in response to a questionnaire displayed on, for instance, the kiosk 110, or on a display device associated with the client device 170 (display device not shown in FIG. 1), and a questionnaire response may be received via user input. The questionnaire response can be transmitted to the response module 310 of the standing orders engine 142.

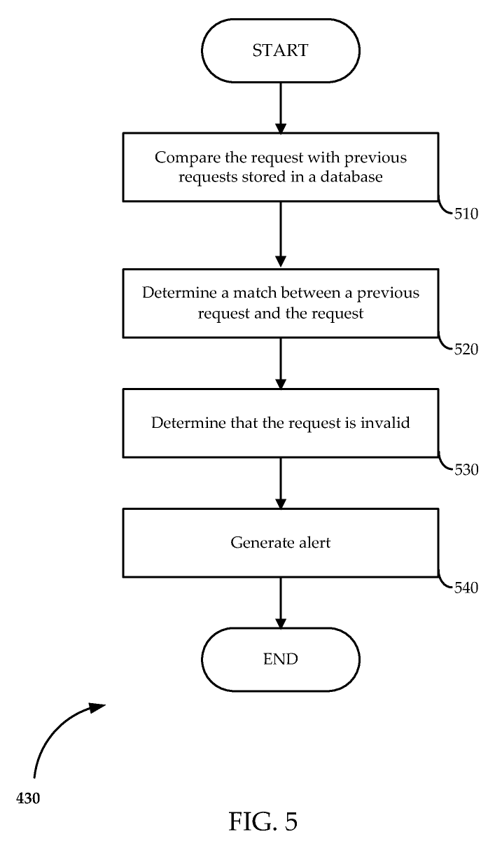

In step 430, the request is processed based on the prescription requirements. The request may be processed by, for instance, the response validation module 315. Step 430 is further discussed in the context of FIG. 5.

In step 440, a prescription is issued to the patient if a determination has been made in step 430 that the questionnaire response satisfies standing order requirements. The prescription can be provided to a user of standing orders engine 142, e.g., a patient or an authorized prescriber of the prescription product. The prescription can be issued by prescription module 330 of the standing orders engine 142 and communicated to, for instance, the client devices 160 and 170. The prescription can be issued based on a generic template or a template specified by the authorized prescriber. The templates may be stored in the data store 150 as discussed above.

FIG. 5 is a flow chart of an exemplary computer-implemented method for automatically determining whether a user response satisfies a standing order in accordance with the methods discussed in the context of FIG. 4. The method 430 disclosed in FIG. 5 can be practiced in the networking environment 100 via elements discussed in the context of FIGS. 1-3 above, for instance, such as by the response module 310 and/or by the response validation module 315 shown in FIG. 3.

In step 510, one or more user requests, e.g., patient requests, are located in the data store 150. These patient requests may correspond to a patient's previous attempts to obtain a prescription automatically by the standing orders engine 142. As discussed above, the patient request may be parsed and/or otherwise processed to locate patient identifiers in the patient request, for instance, a patient name, or an identifier of an authorized prescriber. A search key may be formulated from the aforementioned identifiers and a search of issued prescriptions and/or previously received patient requests stored in the data store 150 may be performed. In some embodiments, the search may be limited only to data stored in association with patients' account information.

If the search of the data store 150 yields one or more previously-stored patient requests, then response module 310 may, in step 520, execute instructions to retrieve and compare the received patient request with those patient requests identified in the search. In some embodiments, the response module 310 may attempt to match the received patient request with the previously stored patient requests. The criteria for determining a match may be set by an authorized prescriber or a system administrator of the standing orders engine 142. For instance, a match may be determined if the previously-stored request and the received request are for the same prescription product and both include the same patient identifiers such as the same patient name, etc.

If a match is determined, in step 530, the received patient request may be determined to be invalid. The response module 310 may transmit a denial notification of the patient request to the client device 170. Additionally, the response module 310 may document the denial of the patient request. For instance, denial of the patient request may be tracked as an event in a historical action log stored in the data store 150. The event may be accessible to the response module 310, for instance, during searches of the data store 150 as discussed in the context of FIG. 3.

The response module 310 may, in step 540, generate an alert upon the determination of invalidity from step 530. The alert may be transmitted to the client device 170, thereby notifying the patient that they are considered ineligible to automatically obtain a prescription from the standing orders engine 142. Alerts may also be transmitted to the authorized prescriber at the client device 160.

In some embodiments, a patient may try to access a prescription multiple times. The present technology may detect multiple attempts by a patient to have a prescription issued, and in some cases reject the user's requests. In some embodiments, the patient may be attempting to refill a prescription, or may be re-visiting a questionnaire when previous information wasn't known. The present technology may allow a patient to request a prescription multiple times if the additional requests are for prescriptions refills, the patient is completing a prior questionnaire, or other reasons that are not related to committing fraud or obtaining a prescription when the patient is not entitled to one.

FIG. 6 is a flow chart of an exemplary method for selecting a questionnaire matching patients with healthcare providers to facilitate a prescription. The method 600 disclosed in FIG. 6 can be practiced in the networking environment 100 via elements discussed in the context of FIGS. 1-3 above, such as by the questionnaire module 325.

Patient information and a request for a prescription product are received at step 610. The patient information and request for prescription product may correspond to the patient request described in the context of FIG. 3 above. The patient information can be received by the response module 310 of the standing orders engine 142. A variety of marketing methods may be used to drive prospective patients to a website, toll-free phone numbers, or some other means of ordering a prescription product, each of which may result in the patient request or other communication being delivered to the response module 310. The information received may include patient identification information, the desired prescription, geographical information for the patient, and other information.

Authorized prescribers that match the identified patient and the desired prescription are determined from the data store 150, and a list of the matching authorized prescribers is provided to the patient at step 620. To determine the matching authorized prescribers, the geographic location of the prospective patient is determined and compared with a database of affiliated authorized prescribers that may provide the desired prescription product in that geographical area.

The patient is then provided with a list of several such authorized prescribers, based on their geographic proximity and on the authorized prescribers' stated geographic proximity requirements. The list of authorized prescribers may be provided to the patient via the kiosk 110, telephone, a webpage, or some other medium suitable to the patient.

A patient selection of one or more of the matching authorized prescribers is received from the patient at step 630. For example, the selection may be received as input of an authorized prescriber from the provided list to the kiosk 110, input received from a client device 170, or some other device and transmitted to the application server 140.

Upon receiving the selection of an authorized prescriber, a standing order associated with the selected authorized prescriber is accessed and the patient is queried for data associated with the standing order at step 640. The query may involve a questionnaire associated with the standing order and provided to the patient through a kiosk such as the kiosk 110, website, patient device, or some other interface. The questionnaire response will provide patient data considered in determining whether to issue the prescription to the patient.

The questionnaire response (e.g., answers to the list of questions associated with the standing order) is received at step 650. A determination is then made as to whether the questionnaire response satisfies the standing order at step 660. As discussed in the context of FIG. 3, the questionnaire provided to the patient may indicate criteria required from a patient in order to qualify for a prescription for the prescription product. As such, appropriate questionnaire responses that provide information as to symptoms, age, or other data, may be necessary to satisfy a standing order. To make the determination, the patient request is compared to the criteria specified in the standing order to see if all, or a satisfactory level of, the criteria are met by the patient data.

If the patient data satisfies the standing order at step 660, the prescription is issued to the patient according to the standing order. Once patients receive a prescription for the prescription product, the prescription product may be dispensed to the patient. Dispensing of the prescription may occur by transmitting the prescription to a pharmacy, transmitting it to the patient such that the patient could, in turn, provide it to a pharmacy, dispensing through kiosk 110 or vending machine 180, or by allowing an order to be processed at a fulfillment center. The standing order, or records associated with the standing order, may be updated to reflect that a prescription was issued, and such information may be stored in the data store 150 in conjunction with the accounts module 335.

If the patient data does not satisfy the standing order criteria at step 660, a determination is made as to whether additional information is required in order to issue the prescription. If additional information is needed and/or insufficient information is available regarding the patient, more information may be acquired from the patient at step 680.

A patient from whom more information is needed may be provided additional instructions on how to continue to interact with the authorized prescriber. These options could include e-mail, a phone call, instant messaging chat, or other electronic communication initiated and/or conducted through the kiosk 110, mobile device 190, or website at client device 170. The patient may also be directed to communicate with an authorized prescriber in person. At any time, the authorized prescriber may reclassify the patient from “Tentative” to “Approved,” at which point, the patient receives a prescription for the prescription product.

If additional information is not needed from the patient, the patient request is denied. Hence, it may be determined that it would not be appropriate to prescribe the prescription product to the patient and alternative recommendations may be provided.

The above-described functions and/or methods may include instructions that are stored on storage media. The instructions can be retrieved and executed by a processor. Some examples of instructions are software, program code, and firmware. Some examples of storage media are memory devices, tape, disks, integrated circuits, and servers. The instructions are operational when executed by the processor to direct the processor to operate in accord with the invention. Those skilled in the art are familiar with instructions, processor(s), and storage media. Exemplary storage media in accordance with embodiments of the invention are illustrated in FIG. 1, which may include, but is not limited to any of components 110-190.

Upon reading this paper, it will become apparent to one skilled in the art that various modifications can be made to the systems, methods, and media disclosed herein without departing from the scope of the disclosure. As such, this disclosure is not to be interpreted in a limiting sense but as a basis for support of the appended claims.

What is claimed is:

1. A method for processing a standing order by a server, comprising:
receiving a standing order by a server, the standing order associated with an authorized prescriber and including prescription requirements for a prescription product;
receiving a request initiated by a user for the prescription product via a remote computing device;
processing the received request by the server based on the prescription requirements; and
automatically issuing a prescription for the prescription product for the user based on the processed request.

2. The method of claim 1, wherein the request includes a result of a questionnaire.

3. The method of claim 1, wherein the received standing order includes geographical information for the authorized prescriber associated with the standing order.

4. The method of claim 3, wherein processing the request includes matching geographical information from the user request to geographical information for the authorized prescriber.

5. The method of claim 1, wherein the received standing order includes expiration data for the standing order.

6. The method of claim 1, wherein processing the request includes determining a location from which to dispense the prescription product to the user.

7. The method of claim 1, further comprising storing the user request in association with the issued prescription.

8. The method of claim 1, wherein processing the received request includes comparing the user request with previous requests by the user stored in the database.

9. The method of claim 8, wherein processing the received request further includes determining whether the request satisfies the prescription requirements based on the number of matching previous requests for the prescription product by the user.

10. The method of claim 1, further comprising updating an inventory associated with the standing order upon issuing the prescription.

11. The method of claim 1, wherein receiving the standing order includes validating the authorized prescriber associated with the standing order.

12. The method of claim 1, wherein receiving the standing order includes receiving a request to purchase the prescription product.

13. The method of claim 11, wherein validating the authorized prescriber includes receiving a valid response to a questionnaire from the authorized prescriber.

14. The method of claim 11, wherein receiving the standing order includes generating a user account associated with the validated authorized prescriber.

15. The method of claim 11, further comprising transmitting a message to the validated authorized prescriber, the message including the issued prescription.

16. The method of claim 11, further comprising transmitting a message to the validated authorized prescriber, the message including the result of the health questionnaire.

17. The method of claim 1, further transmitting a message to the user, the message including the issued prescription.

18. A computer readable storage medium having embodied thereon a program, the program being executable by a processor to perform a method for issuing a prescription, comprising:
receiving a standing order including prescription requirements for a prescription product;
receiving a request from a user for the prescription product, the request including a result of a health questionnaire;
comparing the prescription requirements of the standing order with the result of the health questionnaire; and
issuing a prescription for the prescription product for the user from the standing order based on the comparison.

19. The method of claim 18, wherein issuing the prescription for the prescription product includes storing the user request in association with the issued prescription.

20. The method of claim 18, further comprising receiving a second request from the user for the prescription product.

21. The method of claim 20, further comprising generating an alert upon receiving the second request.

22. The method of claim 20, further comprising comparing the second request with the stored request based on the prescription requirements.

23. A device for issuing a prescription, comprising:
a network interface configured to receive a standing order, the standing order including prescription requirements for a prescription product;
an input interface configured to receive a request from a user for the prescription product via user input to the device, the request including a result of a health questionnaire;
memory; and
a processor configured to execute instructions stored in the memory to compare the prescription requirements of the standing order with the result of the health questionnaire and to issue a prescription for the prescription product for the user from the standing order based on the comparison.
